(19) United States
(12) Patent Application Publication
(10) Pub. No.: US 2024/0148842 A1
(43) Pub. Date: May 9, 2024

(54) PROTEOLYTIC ENZYME MIXTURE FOR TREATING PSORIASIS

(71) Applicant: MEDIWOUND LTD., Yavne (IL)

(72) Inventors: Lior ROSENBERG, Omer (IL); Eilon ASCULAI, Lehavim (IL)

(73) Assignee: MEDIWOUND LTD., Yavne (IL)

(21) Appl. No.: 18/548,385

(22) PCT Filed: Mar. 1, 2022
(86) PCT No.: PCT/IL2022/050224

§ 371 Date: Aug. 30, 2023

Related U.S. Application Data
(60) Provisional application No. 63/154,822, filed on Mar. 1, 2021.

(51) Int. Cl. A61K 38/48; A61K 9/00; A61K 47/02; A61K 47/10; A61K 47/36; A61P 17/06

(52) U.S. Cl. CPC A61K 38/4873; A61K 9/0014; A61K 47/02; A61K 47/10; A61K 47/36; A61P 17/06; C12Y 304/22031; C12Y 304/22032

(57) ABSTRACT

The present invention provides methods of treating cutaneous manifestations of psoriasis comprising topically applying to an affected skin area of a subject a pharmaceutical composition comprising a partially purified proteolytic enzyme mixture obtained from crude bromelain and a pharmaceutically acceptable carrier, thereby treating the cutaneous manifestations of psoriasis.

### FIELD OF THE INVENTION

The present invention relates to pharmaceutical compositions comprising a proteolytic enzyme mixture obtained from crude bromelain for use in treating psoriasis. Particularly, the present invention relates to methods of treating cutaneous manifestations of psoriasis comprising topically applying to an affected skin area of a subject a pharmaceutical composition comprising a partially purified proteolytic enzyme mixture obtained from crude bromelain and a pharmaceutically acceptable carrier, thereby treating one or more cutaneous manifestations of psoriasis.

### BACKGROUND OF THE INVENTION

Psoriasis is a chronic, noncontagious autoimmune disease characterized by raised areas of abnormal skin. These areas vary from discrete erythematous papules and plaques covered with silvery scales, to scaly itching patches that bleed when the scales are removed. The skin lesions in this chronic disease are typically subject to remissions and exacerbations. Psoriasis varies in severity from small, localized patches to complete body coverage.

Psoriasis is generally thought to be a genetic disease that is triggered by environmental factors. It is estimated to affect 2-4% of western world population. It can occur at any age, although it most commonly appears for the first time between the ages of and 25 years.

Psoriasis afflicts the epidermal skin layers. Five layers of the epidermis exist: the stratum basale, the stratum spinosum, the stratum granulosum, the stratum lucidum, and the stratum corneum, the latter is the outermost layer of the epidermis. These different skin layers routinely renew through a natural process known as desquamation, wherein the deepest level skin cells are pushed toward the skin surface. While undergoing desquamation, normal and healthy squamous cells located in the stratum basale divide and begin to produce the keratin that will eventually dominate their contents as the cells are pushed towards the stratum corneum.

Psoriasis is the result of a phenomenon whereby the deepest cells proliferate too rapidly. Healthy skin, and the normal structured layers of the epidermis, cannot shed these hyperplastic proliferating cells quickly enough so the hyperplastic cells accumulate and form thick, dry patches known as plaques. Furthermore, the epidermal skin layers become highly inflamed due to the overpopulation of cells and the movement of immune cells, e.g., dendritic cells, macrophages, and T cells, from the dermis to the epidermis where they secrete cytokines such as interleukin-36γ, tumor necrosis factor-α, interleukin-1β, interleukin-6, and interleukin-22. These secreted inflammatory signals are believed to stimulate keratinocytes to proliferate. The normal epithelium is therefore severely disrupted with the overproduction of these hyperplastic cells. In psoriasis, skin cells are replaced every 3-5 days rather than the usual 28-30 days.

There are five main types of psoriasis: plaque psoriasis, guttate psoriasis, inverse psoriasis, pustular psoriasis, and erythrodermic psoriasis. Plaque psoriasis, also known as psoriasis vulgaris, makes up about 90% of cases. It typically presents as red patches with white scales on top. Areas of the body most commonly affected are the back of the forearms, shins, navel area, and scalp.

No cure for psoriasis is known, but various treatments can help control the symptoms, though they do not provide efficacy and safety that would be considered ideal. These treatments include topical therapy with creams or ointments containing corticosteroids, vitamin D3 analogs, or calcineurin inhibitors; phototherapy with sunlight or ultraviolet light; and oral or injected medications, such as steroids, retinoids, or immunosuppressive drugs, such as methotrexate or cyclosporine.

Corticosteroid creams or ointments are the most frequently prescribed medications for treating mild to moderate psoriasis. Mild corticosteroid ointments (hydrocortisone) are usually recommended for sensitive areas, such as face or skin folds, and for treating widespread patches. Topical corticosteroids might be applied once a day during flares, and on alternate days or weekends only to maintain remission. Triamcinolone or clobetasol creams or ointments are prescribed for smaller, less-sensitive or tougher-to-treat areas. Long-term use or overuse of strong corticosteroids can thin the skin, and may develop permanent stretch marks (striae), bruising, discoloration, and thin spidery blood vessels (telangiectasias). Topical steroids may have a systemic adverse effect, trigger or worsen other skin disorders such as acne, rosacea and perioral dermatitis. Over time, topical corticosteroids can become ineffective.

Creams or ointments containing synthetic forms of vitamin D3, such as calcipotriene and calcitriol, slow skin cell growth. Vitamin D analogs can be used alone or with topical corticosteroids. Calcipotriene and calcitriol are usually less effective than topical corticosteroids.

Retinoids are also used. For example, tazarotene is available as a gel or cream and applied once or twice daily. The most common side effects of tazarotene are skin irritation and increased sensitivity to light. Tazarotene is absorbed through the skin and can be associated with systemic adverse effects including cheilitis, dryness of mucosa and skin, hair loss, photosensitivity, skin fragility, muscle and joint pains, increased intra-cranial pressure, and increased blood lipids. Tazarotene is not recommended for pregnant, breast-feeding, and intend to become pregnant women.

Calcineurin inhibitors, such as tacrolimus and pimecrolimus, reduce inflammation and plaque buildup. They can be especially helpful in areas of thin skin, such as around the eyes, where steroid creams or retinoids are too irritating or may cause harmful effects. Calcineurin inhibitors are absorbed through the skin and can be associated with systemic adverse effects including tremor, nausea, vomiting, burning or itching of the affected skin, stomach upset, acne, muscle or back pain, eye redness/pain, folliculitis, insomnia, hypertension, blurred vision, anemia, hypophosphatemia, asthenia, upper respiratory tract infection, cough, and nephrotoxicity. Calcineurin inhibitors are not recommended for pregnant, breast-feeding, or intend to become pregnant women. It is not intended for long-term use because of a potential increased risk of skin cancer and lymphoma.

U.S. Pat. No. 5,560,910 discloses compositions and methods for topically treating inflammation, wherein the compositions comprise bromelain, capsaicin, and a penetrating agent, preferably n-decylmethyl sulfoxide and/or lecithin organogel.

U.S. Pat. No. 7,731,958 discloses methods for increasing IL-8 level in an individual comprising administering to the individual a composition comprising bromelain. According to U.S. Pat. No. 7,731,958, bromelain can be heat-treated bromelain as the stimulating activity of bromelain on IL-8 level is attributed to non-protease component(s) of bromelain.

U.S. Pat. Nos. 9,821,040 and 10,568,944 disclose compositions containing crude bromelain for use as topical therapeutic agents to restore healthy skin, and for immediate and extended relief from itching and irritation associated with contact dermatitis, insect bites, idiopathic itch, chronic itch, hives, psoriasis, seborrhea, eczema, and more.

U.S. Pat. Application No. 2019/0047769 discloses topical formulations of proteases, preferably bromelain, and devices including the formulations.

WO 2006/054309 to the applicant of the present invention discloses a debriding composition obtained from bromelain comprising most of the proteolytic enzymes present in bromelain and its use for debriding non-viable tissues.

WO 2013/011514 to the applicant of the present invention discloses a proteolytic extract obtained from bromelain for the treatment of connective tissue diseases, in particular Dupuytren's disease and Peyronie's disease.

WO 2017/130204 to the applicant of the present invention discloses debriding compositions comprising a proteolytic enzyme mixture obtained from bromelain in the form of an aqueous gel useful for debridement and treatment of wounds.

WO 2017/183018 to the applicant of the present invention discloses methods of debridement of chronic wounds comprising topically applying to a wound site a debriding formulation in the form of a hydrogel comprising a proteolytic enzyme mixture obtained from bromelain and a water-soluble gelling agent, the debriding formulation being applied to the wound site up to ten times over a period of up to four weeks, thereby achieving debridement of chronic wounds

There still remains an unmet need for improved methods for treating psoriasis lesions which avoid adverse effects.

### SUMMARY OF THE INVENTION

The present invention provides methods of treating at least one cutaneous manifestation of psoriasis comprising topically applying to an affected skin area of a subject a pharmaceutical composition comprising a therapeutically effective amount of a partially purified proteolytic enzyme mixture obtained from crude bromelain and a pharmaceutically acceptable carrier, thereby treating the at least one cutaneous manifestation of psoriasis.

The present invention discloses for the first time that applying topically a pharmaceutical composition comprising a partially purified proteolytic enzyme mixture obtained from crude bromelain, in a form of a hydrogel, to psoriasis lesions resulted in eliminating epidermal plaque thickening, scaling, and flaking. The skin restoration activity of the pharmaceutical composition of the present invention was observed after few topical applications, typically of 3-6 applications, during a short period of time, i.e., a two-week period. The skin regained more healthy and attractive appearance within a month after the beginning of the treatment, and two to three months after the beginning of the treatment, the skin showed fully healthy skin appearance.

It is now disclosed that the pharmaceutical composition of the present invention had a long-term effect, lasting months after cessation of treatment, during which topical corticosteroids or any other anti-psoriasis medications were not required.

It is further disclosed that the pharmaceutical compositions of the present invention are essentially devoid of adverse effects. Importantly, the methods of the present invention avoid the adverse effects which can arise by the use of corticosteroids, retinoids, and/or calcineurin inhibitors, currently being used for topical treatment of psoriasis. The pharmaceutical compositions of the present invention are therefore useful for women and men of all ages who suffer from psoriasis, and particularly for pregnant women, breast-feeding women, and women who intend to be pregnant, whom the use of the common topical medications for treating psoriasis, such as creams containing retinoids, e.g., tazarotene, or calcineurin inhibitors, is contraindicated due to their possible risk to the woman, fetus, and/or newborn.

By virtue of the short-term treatment, the long-term clinical effect, the absence of detectable adverse effects, and its simple use, the pharmaceutical composition of the present invention is highly advantageous over the common topical creams or ointments currently available for treating the cutaneous manifestations of psoriasis.

According to a first aspect, the present invention provides a method of treating one or more cutaneous manifestations of psoriasis comprising topically applying onto an affected skin area of a subject in need of such treatment a pharmaceutical composition comprising a therapeutically effective amount of a partially purified proteolytic enzyme mixture obtained from crude bromelain and a pharmaceutically acceptable carrier, wherein the method avoids adverse effects arising from the use of corticosteroids, retinoids, and/or calcineurin inhibitors known to treat psoriasis.

According to some embodiments, the partially purified proteolytic enzyme mixture is substantially devoid of phosphatases, peroxidases and/or cellulases. According to a certain embodiment, the partially purified proteolytic enzyme mixture is substantially devoid of phosphatases, peroxidases and cellulases.

According to further embodiments, the pharmaceutical composition is devoid of capsaicin.

According to yet further embodiments, the pharmaceutical composition is devoid of an oil and/or a fatty acid.

According to some embodiments, the one or more cutaneous manifestations of psoriasis are selected from the group consisting of epidermal plaque thickening, red patches covered with white scaly skin, pus-filled blisters, dry and itchy cracked skin, and combinations thereof. Each possibility represents a separate embodiment of the invention. According to further embodiments, the adverse effects arising from the use of corticosteroids, retinoids, and/or calcineurin inhibitors are selected from the group consisting of skin thinning, permanent stretch marks (striae), skin discoloration, thin spidery blood vessels, cheilitis, dryness of mucosa and skin, hair loss, photosensitivity, skin fragility, burning or itching of the affected skin, muscle pains, joint pains, increased intra-cranial pressure, increased blood lipids, tremor, nausea, vomiting, stomach upset, eye redness/pain, folliculitis, insomnia, hypertension, blurred vision, anemia, hypophosphatemia, asthenia, upper respiratory tract infection, nephrotoxicity, and combinations thereof. Each possibility represents a separate embodiment of the invention. According to yet further embodiments, the subject is not amenable or is non-responsive to treatment with corticosteroids. According to one exemplary embodiment, the subject is not amenable to treatment with hydrocortisone, triamcinolone and/or clobetasol. According to a certain embodiment, the subject is not amenable to treatment with hydrocortisone, triamcinolone and clobetasol. According to another exemplary embodiment, the subject is non-responsive to treatment with hydrocortisone, triamcinolone and/or clobetasol. According to a certain embodiment, the subject is non-responsive to treatment with hydrocortisone, triamcinolone and clobetasol.

According to some embodiments, retinoids and calcineurin inhibitors are contraindicated for the subject. According to a certain embodiment, the subject is a pregnant woman, a breast-feeding woman, or intend to become pregnant woman.

According to additional embodiments, the pharmaceutical composition is topically applied onto the affected skin area for a duration of about 1 to about 12 hours for 1 to 10 applications or as required to treat said one or more cutaneous manifestations of psoriasis. According to a certain embodiment, the pharmaceutical composition is topically applied onto the affected skin area for up to 6 applications.

According to further embodiments, the pharmaceutical composition is topically applied onto the affected skin area once every day, once every 2 days, once every 3 days, once every 4 days, once every 5 days, once every 6 days, once every 7 days, or any combinations thereof. According to exemplary embodiments, the pharmaceutical composition is topically applied onto the affected skin area for a duration of about 1 to about 4 hours per application, once every 2-5 days, for 3 to 5 applications.

According to some embodiments, the method further comprises a step of topically applying the pharmaceutical composition onto the affected skin area once, twice, trice, or more as needed, during a period of about 3-12 months from the beginning of treatment.

According to certain embodiments, the pharmaceutical composition is applied onto the affected skin area once or twice during a period of about 6 months from the beginning of treatment.

According to some embodiments, the partially purified proteolytic enzyme mixture comprises stem bromelain (EC 3.4.22.32) and ananain (EC 3.4.22.31), and optionally jacalin-like lectin and/or bromelain inhibitors. According to an exemplary embodiment, the partially purified proteolytic enzyme mixture comprises stem bromelain (EC 3.4.22.32), ananain (EC 3.4.22.31), jacalin-like lectin, and bromelain inhibitors.

According to additional embodiments, the amount of the partially purified proteolytic enzyme mixture, also denoted throughout the specification and claims active pharmaceutical ingredient (API), ranges from about 0.1% to about 3% (w/w) of the total weight of the pharmaceutical composition. According to further embodiments, the amount of the partially purified proteolytic enzyme mixture ranges from about 0.5% (w/w) to about 2% (w/w) of the total weight of the pharmaceutical composition. According to still further embodiments, the amount of the partially purified proteolytic enzyme mixture ranges from about 0.6% to about 1.5% (w/w) of the total weight of the pharmaceutical composition. According to an exemplary embodiment, the amount of the partially purified proteolytic enzyme mixture is about 1.25% (w/w) of the total weight of the pharmaceutical composition.

According to some embodiments, the pharmaceutical composition further comprises a water-soluble gelling agent. According to further embodiments, the water-soluble gelling agent is selected from the group consisting of naturally occurring gelling agents, semi-synthetic gelling agents, synthetic gelling agents, and any combinations thereof. Each possibility represents a separate embodiment of the invention. According to yet further embodiment, the water-soluble naturally occurring gelling agent is a polysaccharide. According to still further embodiments, the polysaccharide is a galactomannan, glucomannan or a combination thereof. According to exemplary embodiments, the galactomannan is guar gum present in an amount ranging from about 0.25% (w/w) to about 5% (w/w) of the total weight of the pharmaceutical composition.

According to some embodiments, the pharmaceutical composition further comprises a bulking agent. According to further embodiments, the bulking agent is an oligosaccharide. According to yet further embodiments, the oligosaccharide is selected from the group consisting of lactose, sucrose, mannitol, glucose, and any combinations thereof. Each possibility represents a separate embodiment of the invention. According to exemplary embodiments, the oligosaccharide is lactose present in an amount ranging from about 10% (w/w) to about 25% (w/w) of the total weight of the pharmaceutical composition.

According to some embodiments, the pharmaceutical composition further comprises a pH adjusting agent. According to further embodiments, the pH adjusting agent is potassium phosphate present in an amount ranging from about 2% (w/w) to about 10% (w/w) of the total weight of the pharmaceutical composition. According to yet further embodiments, the potassium phosphate is a combination of potassium phosphate dibasic and potassium phosphate monobasic.

According to some embodiments, the pH of the pharmaceutical composition ranges from about 6.0 to about 8.0. According to a certain embodiment, the pH of the pharmaceutical composition ranges from about 6.0 to about 7.0.

According to additional embodiments, the pharmaceutically acceptable carrier is water present in an amount ranging from about 55% (w/w) to about 90% (w/w) of the total weight of the pharmaceutical composition.

According to some embodiments, the pharmaceutical composition comprises:
(i) the proteolytic enzyme mixture in an amount ranging from about 0.1% (w/w) to about 3% (w/w) of the total weight of said pharmaceutical composition;
(ii) guar gum in an amount ranging from about 0.25% (w/w) to about 5% (w/w) of the total weight of the pharmaceutical composition;
(iii) lactose in an amount ranging from about 10% (w/w) to about 25% (w/w) of the total weight of the pharmaceutical composition;
(iv) potassium phosphate in an amount ranging from about 2% (w/w) to about 10% (w/w) of the total weight of the pharmaceutical composition; and
(v) water in an amount to complete to 100% (w/w) of the total weight of the pharmaceutical composition.

According to a certain embodiment, the pharmaceutical composition comprises:

| Ingredient | (%) w/w of formulation |
|---|---|
| API | 0.5-1.5 |
| Guar gum | 3.5 |
| Lactose | 20.05 |
| Potassium phosphate dibasic | 2.5 |
| Potassium phosphate monobasic | 0.8 |
| Water for injection | To complete to 100 |

According to some embodiments, the partially purified proteolytic enzyme mixture is present in a form of a powdered composition, and prior to use, the powdered composition is admixed with the pharmaceutically acceptable carrier to form the pharmaceutical composition. According to additional embodiments, the powdered composition further comprises said gelling agent, and optionally further comprises said pH adjusting agent, said bulking agent, or a combination thereof. According to a certain embodiment, the powdered composition further comprises said gelling agent, said pH adjusting agent, and said bulking agent, and prior to use, the powdered composition is admixed with water to form a hydrogel, preferably a homogenous hydrogel.

According to another aspect, the present invention provides a pharmaceutical composition comprising a partially purified proteolytic enzyme mixture obtained from crude bromelain and a pharmaceutically acceptable carrier for use in treating one or more cutaneous manifestations of psoriasis by topical application of the pharmaceutical composition onto an affected skin area of a subject.

These and other embodiments of the present invention will be better understood in relation to the description, examples and claims that follow.

### BRIEF DESCRIPTION OF THE FIGURES

FIGS. 1A-H show photographs of psoriasis lesions prior to or post treatment with a hydrogel comprising a partially purified proteolytic enzyme mixture obtained from crude bromelain; the hydrogel is referred herein after as “the formulation”. FIGS. 1A-B show photographs of a skin area affected with psoriasis before or after the P t treatment with the formulation. FIGS. 1C-D show photographs of the skin area affected with psoriasis before or after the 2n d treatment with the formulation. FIGS. 1E-F show photographs of the skin area affected with psoriasis before or after the 4^{th} treatment with the formulation. FIGS. 1G-H show photographs of the skin area of FIGS. 1C-D at day 57 and day 77, respectively, after beginning of treatment, where the skin received 5 treatments with the formulation and then was treated daily with an ointment until day 57 or 77.

### DETAILED DESCRIPTION OF THE INVENTION

The present invention provides pharmaceutical compositions for use in alleviating or treating cutaneous manifestations of psoriasis, wherein the pharmaceutical compositions comprise a partially purified proteolytic enzyme mixture obtained from crude bromelain and a pharmaceutically acceptable carrier.

### Pharmaceutical Compositions

The present invention provides a pharmaceutical composition comprising a partially purified proteolytic enzyme mixture obtained from crude bromelain as an active ingredient, a pharmaceutically acceptable carrier, and one or more excipients.

The term “crude bromelain” refers to a protein extract derived from the stems of pineapple plants which contains a multitude of proteins including enzymes, and polysaccharides. Crude bromelain can be purchased commercially.

The term “proteolytic enzyme mixture partially purified from bromelain” as used herein refers to a mixture comprising proteolytic enzymes fractionated or separated from other non-proteolytic enzymes present in bromelain, e.g., phosphatase(s), peroxidase(s), and/or cellulase(s). The proteolytic enzyme mixture is not a pure proteolytic enzyme preparation. The proteolytic enzyme mixture of the present invention comprises proteolytic enzymes as well as other proteins and constituents as detailed below. However, the proteolytic enzyme mixture is substantially devoid of phosphatases, peroxidases, and cellulases present in crude bromelain.

The term “substantially devoid” in reference to the proteolytic enzyme mixture is meant to indicate that the proteolytic enzyme mixture contains a non-proteolytic enzyme, such as a phosphatase, a peroxidase, and/or a cellulase, at an amount of no more than 20% (w/w) of the amount of that enzyme in crude bromelain prior to fractionation, alternatively of no more than 15% (w/w), 10% (w/w), 5% (w/w), 2% (w/w), or of no more than 1% (w/w) of the amount of the non-proteolytic enzyme in crude bromelain prior to fractionation. Any integer in-between is encompassed. According to a certain embodiment, the proteolytic enzyme mixture is devoid of phosphatases, peroxidases, and cellulases.

The partially purified proteolytic enzyme mixture obtained from crude bromelain (also termed Debrase® or NexoBrid®) and the preparation thereof by partial purification from crude bromelain are disclosed in WO 2006/054309 and WO 2013/011514, the content of which is incorporated by reference as if fully set forth herein. The proteolytic enzyme mixture obtained from crude bromelain comprises at least two of the cysteine proteases present in crude bromelain: stem bromelain (EC 3.4.22.32) and ananain (EC 3.4.22.31). The proteolytic mixture can further comprise one or more of the cysteine protease precursors present in crude bromelain such as, for example, ananain (EC 3.4.22.31) precursor, fruit bromelain (EC 3.4.22.33) precursor, and stem bromelain (EC 3.4.22.31) precursor. The proteolytic enzyme mixture can further comprise cysteine protease fragments (see, for example, WO 2006/054309), a jacalin-like lectin, and/or bromelain inhibitors. According to a certain embodiment, the proteolytic enzyme mixture obtained from crude bromelain comprises stem bromelain (EC 3.4.22.32), ananain (EC 3.4.22.31), a cysteine protease precursor of bromelain, and a jacalin-like lectin.

The proteolytic enzyme mixture can be obtained by the procedure disclosed in WO 2013/011514. As the last step of the preparation, the proteolytic enzyme mixture is lyophilized and stored as a powdered composition until use.

The proteolytic enzyme mixture in its powdered form is highly stable and can be stored at 2-8° C. for long periods of time, e.g., up to three years. After this period of time, the proteolytic enzyme mixture maintains at least 90% of the original proteolytic activity determined prior to its storage.

The proteolytic enzyme mixture is denoted throughout the specification and claims as the active pharmaceutical ingredient (API). According to some embodiments, the amount of API in the pharmaceutical composition ranges from about 0.1% (w/w) to about 3% (w/w) of the total weight of the debriding formulation. According to additional embodiments, the amount of API ranges from about 0.5% (w/w) to about 2% (w/w), such as of about 0.6% (w/w), 0.7%, 0.8%, 0.9%, 1%, 1.1%, 1.2%, 1.3%, 1.4%, or about 1.5% of the total weight of the pharmaceutical composition. According to a certain embodiment, the amount of the API is about 1.25% (w/w) of the total weight of the pharmaceutical composition.

The term “about” refers to ±10% of the value indicated.

The term “powdered” composition as used throughout the specification and claims refers to a composition in a dry or lyophilized form which contains water in an amount of no more than about 5% (w/w) of the total weight of the composition, alternatively of no more than about 3%, 2%, 1%, 0.5%, or further alternatively of no more than about 0.1% (w/w) of the total weight of the composition. According to a certain embodiment, the powdered composition is devoid of water.

The term “hydrogel” as used herein refers to an aqueous composition capable of maintaining a gel-like form. According to some embodiments, the hydrogel is homogenous.

The term “homogenous” hydrogel means a hydrogel having uniform viscosity (e.g., well mixed throughout).

The carrier and excipients of the pharmaceutical composition are all pharmaceutically acceptable. The term “pharmaceutically acceptable” means approved by a regulatory agency of the Federal or a state government or listed in the U. S. Pharmacopeia or other generally recognized pharmacopeia for use in humans.

The term “carrier” refers to a diluent or vehicle with which the active ingredient is administered. Carrier(s) are “acceptable” in the sense of being compatible with the other ingredients of the composition and not deleterious to the recipient thereof. The pharmaceutically acceptable carrier can be water, a dextrose solution, a buffer, or any aqueous solution compatible with the active ingredient. According to an exemplary embodiment, the carrier is water. According to additional embodiments, the carrier is water, further comprising one or more excipients. Alternatively or additionally, the powdered composition can further comprise one or more excipients.

According to some embodiments, the excipients are water-soluble. The term “water soluble” refers to an agent which typically has solubility in water in the range of 1 gr/ml to 1 gr/30 ml at room temperature, i.e., 22-25° C.

The pharmaceutical composition of the present invention can comprise as an excipient a water-soluble gelling agent which can be a naturally occurring gelling agent, a semi-synthetic gelling agent, a synthetic gelling agent, and any combinations thereof. Water-soluble naturally occurring gelling agents include, but are not limited to, water-soluble naturally occurring polysaccharides such as, for example, galactomannans, glucomannans, starches, agar, pectins, alginates, carrageenans, or any combination thereof. Non-limiting examples of galactomannans and glucomannans are guar gum, locust bean gum, xanthan gum, gum acacia, gum tragacanth, gellan gums, and mixtures thereof.

According to an exemplary embodiment, the water-soluble naturally occurring gelling agent is guar gum. According to a certain embodiment, guar gum is present in an amount ranging from about 0.25% (w/w) to about 5% (w/w) of the total weight of the pharmaceutical composition.

Other naturally occurring gelling agents include, for example, chitin, chitosan, glycosaminoglycans such as, for example, heparin, chondroitin sulfate, dermatan sulfate, heparan sulfate, and proteoglycans.

Semi-synthetic gelling agents include, but are not limited to, cellulose ethers (e.g., hydroxyethyl cellulose, methyl cellulose, carboxymethyl cellulose, hydroxy propylmethyl cellulose), polyvinylalcohol, hydroxypropyl guar gum, and the like.

The synthetic gelling agents include, but are not limited to, carboxyvinyl polymers, polyvinylpyrrolidone, polyvinyl acetate polymers, polyvinyl chloride polymers, polyvinylidene chloride polymers, cross-linked polymers of acrylic acid such as carbomers or carbopols, and the like.

The pharmaceutical composition can further comprise as an excipient a bulking agent and/or a pH adjusting agent.

The bulking agents suitable for practicing the present invention is any known bulking agent, such as an oligosaccharide, for example, lactose, sucrose, mannitol, sorbitol, and glucose; an amino acid, for example, glycine. According to a certain embodiment, the bulking agent is lactose. According to one embodiment, lactose is present in an amount ranging from about 10% (w/w) to about 25% (w/w) of the total weight of the pharmaceutical composition.

The pH adjusting agent preferably has a pKa of above 6.0. In some embodiments, the pH adjusting agent can be any known pH adjusting agent such as, for example, potassium phosphate, potassium carbonate, sodium carbonate, and sodium phosphate.

According to some embodiments, the pH adjusting agent is a combination of potassium phosphate monobasic and potassium phosphate dibasic present in an amount ranging from about 2% (w/w) to about 10% (w/w) of the total weight of the pharmaceutical composition.

According to one exemplary embodiment, the pharmaceutical composition comprises or consists of:

| Ingredient | (%) w/w of formulation |
|---|---|
| API | 0.5-1.5 |
| Guar gum | 3.5 |
| Lactose | 20.05 |
| Potassium phosphate dibasic | 2.5 |
| Potassium phosphate monobasic | 0.8 |
| Water for injection | To complete to 100 |

The pharmaceutical composition can further comprise an anti-foaming agent. Anti-foaming agents are known in the art and include, but are not limited to, polyethylene glycols, e.g., PEG-1450, PEG-3350, and the like.

According to another exemplary embodiment, the pharmaceutical composition comprises or consist of:

| Ingredient | (%) w/w of formulation |
|---|---|
| API | 0.5-1.5 |
| Guar gum | 3.5 |
| Lactose | 18.05 |
| Potassium phosphate dibasic | 2.5 |
| Potassium phosphate monobasic | 0.8 |
| PEG-3350 | 2 |
| Water for injection | To complete to 100 |

The composition can further comprise a preservative such as, for example, benzyl alcohol, parabens, methyl- or propylhydroxybenzoates; and/or an anti-oxidant such as, for example, ascorbic acid, dihydroquinone, butylated hydroxytoluene, and dithiothreitol.

The pharmaceutical composition can further comprise an additional active agent selected from the group consisting of anesthetic agents, antibacterial agents, antifungal agents, anti-inflammatory agents, analgesic agents, and agents which promote healing. The anesthetic agents include, but are not limited to, amethocaine (tetracaine), lignocaine (lidocaine), xylocaine, bupivacaine, prilocaine, ropivacaine, benzocaine, mepivocaine, cocaine. Each possibility represents a separate embodiment.

The antibacterial agents include, but are not limited to, amikacin, amikacin sulfate, aminoglycosides, amoxicillin, ampicillin, ansamycins, bacitracin, beta-lactams, capreomycin, carbenicillin, cephalexin, cephaloridine, cephalothin, cefazolin, cephapirin, cephradine, cephaloglycin, chloramphenicols, chlorhexidine, chlorhexidine gluconate, chlorhexidine hydrochloride, chloroxine, chlorquinaldol, chlortetracycline, chlortetracycline hydrochloride, ciprofloxacin, circulin, clindamycin, clindamycin hydrochloride, clotrimazole, cloxacillin, demeclocycline, dicloxacillin, diiodohydroxyquin, doxycycline, ethambutol, ethambutol hydrochloride, erythromycin, erythromycin estolate, erythromycin stearate, farnesol, floxacillin, gentamicin, gentamicin sulfate, gramicidin, griseofulvin, haloprogin, halquinol, hexachlorophene, minocylcline, iodochlorhydroxyquin, kanamycin, kanamycin sulfate, lincomycin, lincomycin, lincomycin hydrochloride, macrolides, meclocycline, methacycline, methacycline hydrochloride, methenamine, methenamine hippurate, methenamine mandelate, methicillin, metronidazole, miconazole, miconazole hydrochloride, minocycline, minocycline hydrochloride, mupirocin, nafcillin, neomycin, neomycin sulfate, netilmicin, netilmicin sulfate, nitrofurazone, norfloxacin, nystatin, oleandomycin, cephalosporins, oxacillin, oxytetracycline, oxytetracycline hydrochloride, parachlorometaxylenol, paromomycin, paromomycin sulfate, penicillins, penicillin G, penicillin V, pentamidine, pentamidine hydrochloride, pheneticillin, polymyxins, quinolones, streptomycin sulfate, tetracycline, tobramycin, tolnaftate, triclosan, rifampin, rifamycin, rolitetracycline, silver salts, spectinomycin, spiramycin, streptomycin, sulfonamide, tetracyclines, tetracycline, tobramycin, tobramycin sulfate, triclocarban, triclosan, trimethoprim-sulfamethoxazole, tylosin, vancomycin, and tyrothricin. Each possibility represents a separate embodiment.

The antifungal agents include, but are not limited to, nystatin, clotrimazole, miconazole, ketoconazole, fluconazole, thiabendazole, econazole, chlormidazole, isoconazole, tiabendazole, tioconazole, sulconazole, bifonazole, oxiconazole, fenticonazole, omoconazole, sertaconazole, and flutrimazole. Each possibility represents a separate embodiment.

The anti-inflammatory agent can be non-steroidal anti-inflammatory agent, steroidal anti-inflammatory agent, or a combination thereof. Non limiting examples of non-steroidal anti-inflammatory agents include oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acemetacin, fentiazac, zomepirac, clindamycin, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Extracts of these non-steroidal anti-inflammatory agents may also be employed. Each possibility represents a separate embodiment.

Non-limiting examples of steroidal anti-inflammatory drugs include corticosteroids such as hydrocortisone, hydroxyl-triamcinolone, alpha-methyl dexamethasone, dexamethasone-phosphate, beclomethasone dipropionates, clobetasol valerate, desonide, desoximetasone, deoxycorticosterone acetate, dexamethasone, dichlorisone, diflorasone diacetate, diflucortolone valerate, fluradrenolone, fluclorolone acetonide, fludrocortisone, flumethasone pivalate, fluocinolone acetonide, fluocinonide, flucortine butyl esters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, fluocinonide, fludrocortisone, diflorasone diacetate, flurandrenolone, fludrocortisone, diflorasone diacetate, fluradrenolone acetonide, medrysone, amcinafal, amcinafide, betamethasone and the balance of its esters, chloroprednisone, chlorprednisone acetate, clorcortolone, clescinolone, dichlorisone, diflurprednate, flucloronide, flunisolide, fluorometholone, fluperolone, fluprednisolone, hydrocortisone valerate, hydrocortisone cyclopentyl propionate, hydrocortamate, meprednisone, paramethasone, prednisolone, prednisone, beclomethasone dipropionate, and triamcinolone. Each possibility represents a separate embodiment.

Analgesic agents include, but are not limited to, codeine, hydrocodone, oxycodone, fentanyl, and propoxyphene. Local analgesic agents include cocaine derivatives including, but not limited to, lidocaine, lidocaine, and pontocaine. Each possibility represents a separate embodiment.

Agents which promote healing include, but are not limited to, hyaluronic acid.

According to some embodiments, the proteolytic enzyme mixture is stored as a powdered composition, and prior to use, the powdered composition is admixed with the pharmaceutically acceptable carrier to form the pharmaceutical composition. According to some embodiments, the powdered composition further comprises a water-soluble gelling agent, and prior to use, said powdered composition is admixed with water or with an aqueous solution to form a hydrogel having the desired pH. According to further embodiments, the powdered composition can further comprise a pH adjusting agent and/or a bulking agent. Alternatively, the pharmaceutically acceptable carrier can comprise a water-soluble gelling agent, a pH adjusting agent and/or a bulking agent.

According to some embodiments, the powdered composition and the pharmaceutically acceptable carrier, such as water, are stored in a first compartment and a second compartment, respectively, of a single container or can be stored in two separate containers. Before use, the powdered composition and the water are admixed to form the pharmaceutical composition in a form of a hydrogel.

According to some embodiments, the pharmaceutical compositions of the present invention are sterile.

According to some embodiments, the pharmaceutical composition comprises:
(a) a powdered composition comprising:
   (i) the partially purified proteolytic enzyme mixture obtained from crude bromelain;
   (ii) a water-soluble gelling agent;
   (iii) a bulking agent;
   (iv) a pH adjusting agent; and
(b) water,
wherein the powdered composition of (a) is admixed with the water of (b) to form a pharmaceutical composition in the form of a hydrogel having a pH ranging from about 6.0 to about 8.0, and wherein the amount of the proteolytic enzyme mixture ranges from about 0.1% (w/w) to about 3% (w/w) of the total weight of the pharmaceutical composition.

According to some embodiments, the pharmaceutical composition comprises:
(a) a powdered composition, present in a first compartment of a container or in a first container, the powdered composition comprising:
   (i) the partially purified proteolytic enzyme mixture obtained from crude bromelain;
   (ii) guar gum in an amount ranging from about 0.25% (w/w) to about 5% (w/w) of the total weight of the debriding formulation;
   (iii) lactose in an amount ranging from about 10% (w/w) to about 25% (w/w) of the total weight of the debriding formulation;
   (iv) a pH adjusting agent; and
(b) water in an amount ranging from about 55% (w/w) to about 90% (w/w), present in the second compartment of the container or in the second container.

The ranges of numerical values indicated throughout the specification and claims include any integer or fraction thereof in-between.

It is to be appreciated that the partially purified proteolytic enzyme mixture of the present invention is substantially devoid of non-proteolytic enzymes, such as phosphatases, peroxidases and/or cellulases. According to a certain embodiment, the partially purified proteolytic enzyme mixture contains no more than 1% of the amount of phosphatases, peroxidases and cellulases present in crude bromelain. According to a certain embodiment, the pharmaceutical composition of the present invention is devoid of phosphatases, peroxidases, cellulases, and capsaicin.

As the proteolytic enzymes obtained from bromelain are water-soluble, addition of an oil and/or a fatty acid is typically avoided.

### Uses

The present invention provides a method of treating one or more cutaneous manifestations of psoriasis comprising topically applying to an affected skin area of a subject in need of such treatment a pharmaceutical composition comprising a therapeutically effective amount of a partially purified proteolytic enzyme mixture obtained from crude bromelain and a pharmaceutically acceptable carrier, wherein the method avoids adverse effects arising from the use of corticosteroids, retinoids, and/or calcineurin inhibitors.

The present invention further provides a method of treating psoriasis comprising topically applying to an affected skin area of a subject in need of such treatment a pharmaceutical composition comprising a therapeutically effective amount of a partially purified proteolytic enzyme mixture obtained from crude bromelain and a pharmaceutically acceptable carrier, thereby treating psoriasis.

The term “cutaneous manifestations” of psoriasis as used herein refers to an abnormal skin appearance, abnormal skin growth, and/or abnormal skin sensation which are associated with psoriasis and include, but are not limited to, raised and dry skin areas also known as “plaque epidermal thickening”, red skin areas covered with white scaly skin, pus-filled blisters, skin flaking, erythema, dry cracked skin, itching, and burning. Cutaneous manifestations of psoriasis are also demonstrated by histological examination of psoriasis lesions. Therefore, cutaneous manifestations of psoriasis include, but are not limited to, hyperproliferative epidermis with elongated rete ridges, altered keratinization, neutrophils in the stratum corneum, and dilated capillaries in the dermis.

The term “therapeutically effective amount” is that amount of the partially purified proteolytic enzyme mixture which is sufficient to provide a beneficial effect to the subject to whom the composition is applied. Thus, a therapeutically effective amount is an amount sufficient to cause, for example, an epidermal plaque thickening to shrink or heal and/or to decrease the growth rate of a plaque (such as to suppress plaque growth) and/or to regain normal, healthy skin appearance.

The terms “treatment” or “treating” as used herein interchangeably refer to obtaining beneficial or desired clinical results, or any measurable mitigation of psoriasis in a subject, including resolution, reduction, halting progression, and/or slowing progression, of the disease. Beneficial or desired clinical results include, but are not limited to, one or more of the following: alleviating one or more symptoms or cutaneous manifestations of psoriasis (such as reducing or arresting plaque growth, skin scaling or flaking, dry cracked skin, bleeding, itching, and burning), diminishing the extent of psoriasis, stabilizing (i.e., not worsening) the state of psoriasis, preventing or delaying recurrence of psoriasis, delaying or slowing psoriasis progression, ameliorating psoriatic state, and regaining normal, healthy skin appearance.

The terms “skin scaling” and “skin flaking” are used interchangeably to refer to loss of the outer layer of the epidermis in large scale-like flakes.

There are five main types of psoriasis: plaque psoriasis, guttate psoriasis, inverse psoriasis, pustular psoriasis, and erythrodermic psoriasis.

Plaque psoriasis, also known as psoriasis vulgaris, makes up about 90% of cases. It typically presents as red patches with white scales on top. Areas of the body most commonly affected are the back of the forearms, shins, navel area, and scalp.

Guttate psoriasis has drop-shaped lesions.

Pustular psoriasis presents as small, noninfectious, pus-filled blisters.

Inverse psoriasis forms red patches in skin folds.

Erythrodermic psoriasis occurs when the rash becomes very widespread, and can develop from any of the other types.

According to some embodiments, the method of the present invention is useful for treating any type of psoriasis. The method is also useful for treating nail psoriasis and/or scalp psoriasis.

The method of the present invention avoids the adverse effects associated with known medications currently being used for treating psoriasis. It is noted that the mild irritation, when caused with the pharmaceutical composition of the present invention, was temporary and disappeared as soon as the pharmaceutical composition was removed from the treated skin, and a moisturizing-emollient cream was applied onto the treated skin. Without being bound to any mechanism of action, it is suggested that the mild irritation is the result of the therapeutic effect, i.e., the proteolytic activity, of the proteolytic enzyme mixture. It is, therefore, not considered an adverse effect.

While topical application of corticosteroids, retinoids, and calcineurin inhibitors on psoriasis lesions can be associated with adverse effects, the pharmaceutical compositions of the present invention avoid the adverse effects arising from the use of these medications. The adverse effects of corticosteroids, e.g., hydrocortisone, triamcinolone, and clobetasol, retinoids, e.g., tazarotene, and calcineurin inhibitors, e.g., tacrolimus and pimecrolimus, include, but are not limited to, skin thinning, permanent stretch marks (striae), skin discoloration, thin spidery blood vessels, cheilitis, dryness of mucosa and skin, hair loss, photosensitivity, skin fragility, burning or itching of the affected skin, muscle pains, joint pains, increased intra-cranial pressure, increased blood lipids, tremor, nausea, vomiting, stomach upset, eye redness/pain, folliculitis, insomnia, hypertension, blurred vision, anemia, hypophosphatemia, asthenia, upper respiratory tract infection, and nephrotoxicity. According to some embodiments, the method of the present invention is useful for treating subjects with psoriasis who are not amenable or are non-responsive to treatment with topical corticosteroids.

The term “non-responsive” refers to subjects with psoriasis who have been treated with a topical corticosteroid, wherein the corticosteroid does not have a beneficial, therapeutic effect.

According to additional embodiments, the method of the present invention is useful for women at childbearing age having psoriasis, particularly for pregnant women, breast-feeding women, and women who intend to be pregnant. Due to the adverse effects which can arise by topical treatment of psoriasis lesions with retinoids and/or calcineurin inhibitors, pregnant women, breast-feeding women, and women who intend to be pregnant, should avoid the use of these medications.

According to some embodiments, the method comprises applying the pharmaceutical composition onto the affected skin area for a duration of about 1 to about 12 hours for 1 to 10 applications, or as required to treat the one or more cutaneous manifestations of psoriasis or to treat psoriasis.

According to additional embodiments, the pharmaceutical composition is topically applied for a duration of about 1 to about 12 hours at a frequency selected from the group consisting of: once a day, once in 2 days, once in 3 days, once in 4 days, once in 5 days, once in 6 days, once a week, twice a month, once a month, and any combinations thereof, for 1 to 10 applications, or as required to treat the one or more cutaneous manifestations of psoriasis or to treat psoriasis.

According to some embodiments, the method comprises applying the pharmaceutical composition for a duration of about 1 to about 4 hours every 2 to 5 days for 1 to 10 applications, or as required to treat the one or more cutaneous manifestations of psoriasis or to treat psoriasis. According to additional embodiments, the method comprises applying the pharmaceutical composition for a duration of 1 to 4 hours every 2 days, 3 days, 4 days, 5 days, or any combination thereof, for 3, 4, 5, 6, or 7 applications. According to certain embodiments, the method comprises applying the pharmaceutical composition onto an affected skin area for a duration of 1 to 4 hours every 3 days for 3, 4 or 5 applications.

The method of the present invention is useful to restore skin of a subject having psoriasis to a more healthy and attractive state, preferably to restore skin at psoriasis affected areas to a normal state.

According to some embodiments, following the application of the pharmaceutical composition, the method further comprises a step of applying an adhesive barrier onto the healthy normal skin, adjacent to the edges of the treated area. The adhesive barrier can be a hydrophobic ointment or paste including, but not limited to, petroleum gels, fatty ointments, zinc oxide pastes, and silicon gels. The adhesive barrier adheres to the healthy normal skin and forms a repellent barrier which prevents the pharmaceutical composition to reach and affect the normal healthy skin.

According to some embodiments, following the application of the pharmaceutical composition, the method can further comprise a step of covering the pharmaceutical composition only, or the pharmaceutical composition and the adhesive barrier, with an occlusive layer or dressing to maintain or hold the composition at the treated site.

According to additional embodiments, the method further comprises removing the pharmaceutical composition after the 1 to 12 hours of application, and applying an emollient. Applying an emollient can be performed once a day, twice a day, or as many times as required to protect or soften the skin.

The term “emollients” as used herein refers to lipids, waxes, fatty acids, or substances with similar consistency, or any combinations thereof, which, when applied to the skin, protect and soften the skin, making it more supple. Emollients are used primarily as excipients or bases of ointments and other dermatological preparations such as, for example, moisturizing creams, and lotions.

Although the methods of the present invention are intended to replace corticosteroids, retinoids, and calcineurin inhibitors for treating psoriasis, the methods of the present invention, according to some embodiments, can be combined with methods known for treating psoriasis, such as, sunlight or UV therapy, topical medicaments, e.g., corticosteroids, vitamin D3 analogs, retinoids, calcineurin inhibitors, coal tar extract, salicylates, or oral or injectable medicaments, e.g., steroids, retinoids, methotrexate, cyclosporine, etanercept, infliximab, adalimumab, ustekinumab, secukinumab, and ixekizumab. According to some embodiments, the methods of the present invention can be performed prior to, simultaneously or after any known treatment method of psoriasis.

According to some embodiments, the amount of API applied ranges between about 0.1 gr to about 2 gr of the sterile lyophilized proteolytic enzyme mixture per 100 cm 2 of psoriatic lesion. According to additional embodiments, the amount of hydrogel applied to a psoriasis site is of about 20 gr per 100 cm 2 of a skin lesion.

It is to be understood that each possibility disclosed throughout the specification represents a separate embodiment of the invention.

### Example 1

### Hydrogel Formulation

A dried formulation which contained the proteolytic enzyme mixture obtained from crude bromelain (API), guar gum, lactose, potassium phosphate monobasic and dibasic, and polyethylene glycol, as detailed in the table below, was mixed, prior to use, with water to obtain a hydrogel which was applied to psoriasis lesions in the experiment described herein below. The hydrogel formed is designated herein below “the formulation”.

TABLE 1
The constituents of the formulation.

| Ingredient | (%) w/w of formulation |
|---|---|
| API | 1.25 or 0.625 |
| Guar gum | 3.5 |
| Lactose | 18.05 |
| Potassium phosphate dibasic | 2.5 |
| Potassium phosphate monobasic | 0.8 |
| PEG-3350 | 2 |
| Water for injection | To complete to 100 |

### Example 2

### Treatment of Psoriatic Lesions

Mild and moderate plaque psoriasis lesions, which were treated unsuccessfully with steroids for 12 years and with phototherapy for 1 year, were treated as follows: the formulation containing 1.25% w/w API was applied for 3-4 hours at days 1, 3, 8 and 11. These days were designated the “Removal phase”, i.e., the first two weeks of treatment. After each treatment with the formulation, the psoriasis lesions were cleaned and wiped, and an emollient moisturizing-nurturing ointment (Aquaphor and Vaseline) was applied. At day 29 (designated herein the “Recovery phase”, i.e., from the 3r d week until 2 months from the beginning of treatment), the formulation containing 1.25% w/w API was applied once for 3-4 hours, and then wiped and an emollient moisturizing-nurturing ointment was applied onto the lesions again. During the “Maintenance phase”, i.e., from the 3^{rd} month until 6 months from the beginning of treatment, the formulation was applied once at a lower amount of API (0.625% w/w). During the Recovery and Maintenance phases, an emollient moisturizing-nurturing ointment was applied daily.

Application of the formulation according to this protocol did not cause detectable adverse effects. It was safe for use. In those few instances where some irritation or pain occurred, the formulation was wiped after 3-4 hours of use and the application of an emollient moisturizing-nurturing ointment stopped the irritation and/or pain.

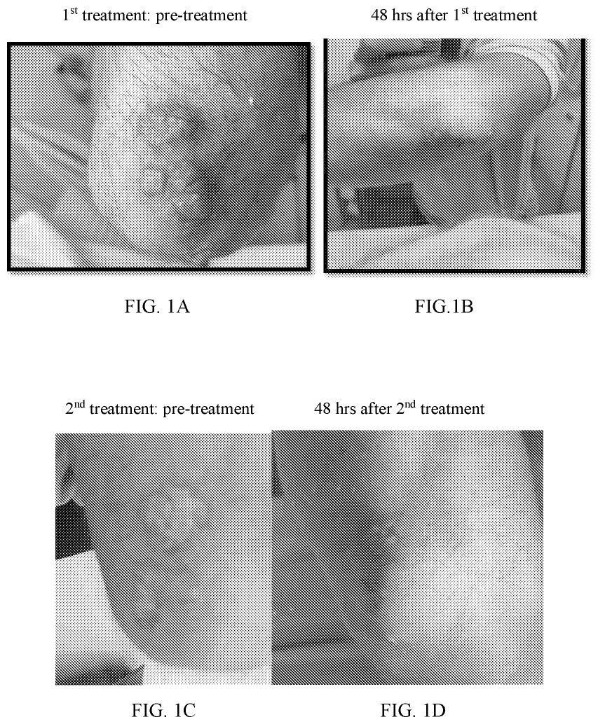

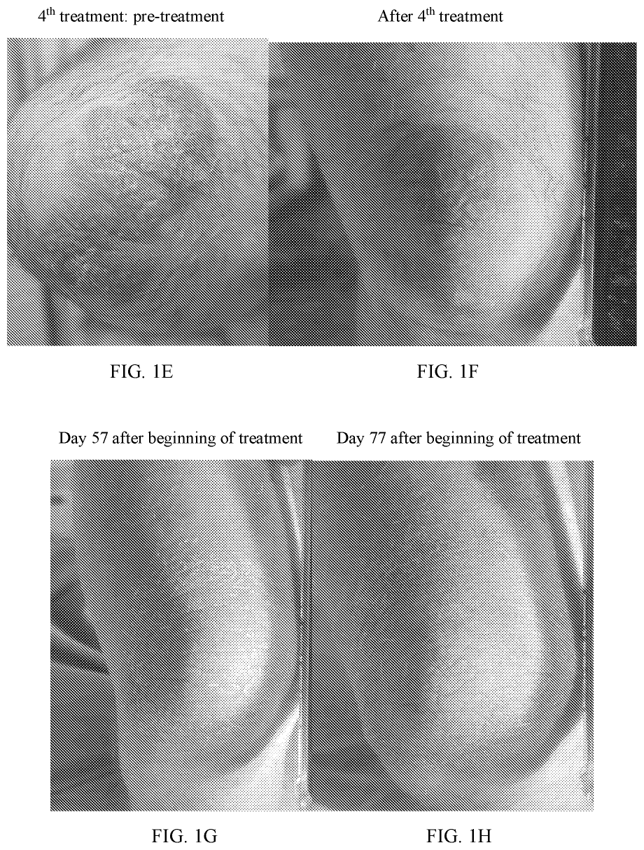

As shown in FIGS. 1A-F, psoriasis lesions treated with the formulation were gradually removed after the 1^{st}, 2^{nd}, 3^{rd}, and clearly after 4^{th} treatments, and the skin after these treatments returned to almost a normal appearance under a maintenance care of an emollient moisturizing-nurturing ointment. Skin appearance improved during the Recovery phase and Maintenance phase, e.g., at day 57 and day 77, respectively, as shown in FIG. 1G and FIG. 1H, respectively. Four months from the beginning of treatment (at day 120 from the first treatment), there was no need to apply the formulation or any specific anti-psoriasis treatment. These results indicated that the formulation was safe as it did not cause detectable adverse effects, and it was highly effective as very few applications at a two-week period were required for treating psoriasis cutaneous manifestations, and hence for regaining healthy skin appearance.

It will be appreciated by persons skilled in the art that the present invention is not limited by what has been particularly shown and described herein above. Rather the scope of the invention is defined by the claims that follow.

1. A method of treating one or more cutaneous manifestations of psoriasis comprising topically applying onto an affected skin area of a subject in need of such treatment a pharmaceutical composition comprising a therapeutically effective amount of a partially purified proteolytic enzyme mixture obtained from crude bromelain and a pharmaceutically acceptable carrier, wherein the method avoids adverse effects arising from the use of corticosteroids, retinoids, and/or calcineurin inhibitors known to treat psoriasis.

2. The method according to claim 1, wherein the partially purified proteolytic enzyme mixture is substantially devoid of phosphatases, peroxidases, cellulases, capsaicin, an oil and/or a fatty acid.

3.-5. (canceled)

6. The method according to claim 1, wherein the one or more cutaneous manifestations of psoriasis are selected from the group consisting of epidermal plaque thickening, red patches covered with white scaly skin, pus-filled blisters, dry and itchy cracked skin, and combinations thereof.

7. The method according to claim 1, wherein the adverse effects arising from the use of corticosteroids, retinoids, and/or calcineurin inhibitors are selected from the group consisting of skin thinning, permanent stretch marks (striae), skin discoloration, thin spidery blood vessels, cheilitis, dryness of mucosa and skin, hair loss, photosensitivity, skin fragility, burning or itching of the affected skin, muscle pains, joint pains, increased intra-cranial pressure, increased blood lipids, tremor, nausea, vomiting, stomach upset, eye redness/pain, folliculitis, insomnia, hypertension, blurred vision, anemia, hypophosphatemia, asthenia, upper respiratory tract infection, nephrotoxicity, and combinations thereof.

8. The method according to claim 1, wherein the subject is not amenable or is non-responsive to treatment with corticosteroids.

9. (canceled)

10. (canceled)

11. The method according to claim 1, wherein retinoids and calcineurin inhibitors are contraindicated for the subject.

12. The method according to claim 11, wherein the subject is a pregnant woman, a breast-feeding woman, or intend to become a pregnant woman.

13. The method according to claim 1, wherein the pharmaceutical composition is topically applied onto the affected skin area for a duration of about 1 to about 12 hours for 1 to 10 applications, or as required, to treat said one or more cutaneous manifestations of psoriasis.

14. (canceled)

15. (canceled)

16. The method according to claim 13, wherein the pharmaceutical composition is topically applied onto the affected skin area for a duration of about 1 to about 4 hours, once every 2-5 days, for 3 to 5 applications.

17. (canceled)

18. (canceled)

19. The method according to claim 1, wherein the partially purified proteolytic enzyme mixture comprises stem bromelain (EC 3.4.22.32) and ananain (EC 3.4.22.31), and optionally jacalin-like lectin and/or bromelain inhibitors, and wherein the amount of the partially purified proteolytic enzyme mixture ranges from about 0.1% to about 3% (w/w) of the total weight of the pharmaceutical composition.

20.-24. (canceled)

25. The method according to claim 1, wherein the pharmaceutical composition further comprises a water-soluble gelling agent selected from the group consisting of naturally occurring gelling agents, semi-synthetic gelling agents, synthetic gelling agents, and any combinations thereof.

26. (canceled)

27. The method according to claim 25, wherein the water-soluble naturally occurring gelling agent is a polysaccharide, and wherein the polysaccharide is a galactomannan, a glucomannan, or a combination thereof.

28. (canceled)

29. The method according to claim 27, wherein the galactomannan is guar gum present in an amount ranging from about 0.25% (w/w) to about 5% (w/w) of the total weight of the pharmaceutical composition.

30. The method according to claim 1, wherein the pharmaceutical composition further comprises a bulking agent.

31. The method according to claim 30, wherein the bulking agent is an oligosaccharide selected from the group consisting of lactose, sucrose, mannitol, glucose, and combinations thereof.

32. (canceled)

33. The method according to claim 31, wherein the oligosaccharide is lactose present in an amount ranging from about 10% (w/w) to about 25% (w/w) of the total weight of the pharmaceutical composition.

34. The method according to claim 1, wherein the pharmaceutical composition further comprises a pH adjusting agent, and wherein the pH of the pharmaceutical composition ranges from about 6.0 to about 8.0.

35.-39. (canceled)

40. The method according to claim 1, wherein the pharmaceutical composition comprises:
(i) the partially purified proteolytic enzyme mixture which comprises stem bromelain (EC 3.4.22.32), ananain (EC 3.4.22.31), jacalin-like lectin, and bromelain inhibitors, in an amount ranging from about 0.1% (w/w) to about 3% (w/w) of the total weight of said pharmaceutical composition;
(ii) guar gum in an amount ranging from about 0.25% (w/w) to about 5% (w/w) of the total weight of the pharmaceutical composition;
(iii) lactose in an amount ranging from about 10% (w/w) to about 25% (w/w) of the total weight of the pharmaceutical composition;
(iv) the pH adjusting agent which is potassium phosphate present in an amount ranging from about 2% (w/w) to about 10% (w/w) of the total weight of the pharmaceutical composition; and
(v) water in an amount to complete to 100% (w/w) of the total weight of the pharmaceutical composition.

41. The method according to claim 40, wherein the pharmaceutical composition comprises:

| Ingredient | (%) w/w of formulation |
|---|---|
| API | 0.5-1.5 |
| Guar gum | 3.5 |
| Lactose | 20.05 |
| Potassium phosphate dibasic | 2.5 |
| Potassium phosphate monobasic | 0.8 |
| Water for injection | To complete to 100 |

42. The method according to claim 1, wherein the partially purified proteolytic enzyme mixture is present in a form of a powdered composition, and prior to use, the powdered composition is admixed with the pharmaceutically acceptable carrier to form the pharmaceutical composition in a form of a hydrogel.

43. (canceled)

44. (canceled)
